(19) United States
(12) Patent Application Publication
(10) Pub. No.: US 2002/0080328 A1
(43) Pub. Date: Jun. 27, 2002

(54) Instruments for examining the body or part thereof

(76) Inventor: Eugene Robert Van Arsdale, Philadelphia, PA (US)

Correspondence Address: Lee, Mann, Smith, McWilliams, Sweeney and Ohlson, Suite 410, 209 S. LaSalle St., Chicago, IL, 60604, US

(21) Appl. No.: 09/948,842

(22) Filed: Sep. 7, 2001

(30) Foreign Application Priority Data
Sep. 9, 2000 (GB) 0022135.8

(51) Int. Cl. A61B 3/00

(52) U.S. Cl. 351/200

(57) ABSTRACT

An indirect ophthalmoscope (or other like instrument) is provided witha detachable cover which fits over the front of the instrument and is shaped so as to allow the instrument to be used with the cover in place. The cover can bear a decorative design so that the ophthalmoscope can be given a less threatening appearance when being used to examine the eyes of children.

### FIELD OF THE INVENTION

This invention relates to an instrument for use in the direct examination of the human body or part thereof. The invention is particularly applicable to an ophthalmic instrument, such as an indirect ophthalmoscope, and to a cover for such an instrument.

### BACKGROUND TO THE INVENTION

A proper examination of an eye with, for example, an indirect ophthalmoscope, typically requires, among other things, that the patient remains still. Moreover, it is often necessary for patients to avoid moving their eyes, and in this connection that patient can be asked to stare at a fixed point with the ophthalmoscope in the patient's field of view.

However, some patients, especially children, become nervous during examination and as a result find it difficult to keep their heads, and eyes, stationary.

In addition, there are a number of adjustments which can be made to an ophthalmoscope to make it suitable for use by a given user. Thus, in busy environments, for example student classes, it can be useful for an individual to be able easily to identify which ophthalmoscope has been set up for them to use, and to avoid choosing an ophthalmoscope set up for somebody else.

### SUMMARY OF THE INVENTION

According to a first aspect of the invention, there is provided an instrument for use in the direct examination of the human body or part thereof, the instrument having a cover so shaped as to allow the instrument to be used with the cover in place, wherein the cover is detachable.

The use of a detachable cover gives the invention a number of advantages, depending upon the nature of the cover used. Where it is important that the user of the instrument (i.e. the person who is using the instrument to examine somebody else's body) is to be able to identify their instrument, the cover can bear one or more distinctive identification indicia, such as a printed pattern or design or distinctive shaped portion. For example, the user's name may be printed on the cover. Additionally or Alternatively, the cover may bear a decorative design.

Preferably, the instrument has a housing which contains one or more components thereof, wherein the cover extends over the front of the housing, when fitted thereon.

If the cover is a decorative cover, the instrument will thus have a decorative appearance to the person being examined.

Preferably, the cover is co-extensive with the housing.

The cover is especially advantageous if the instrument is for use in examining the human face or part thereof, as in this case the instrument may well be held within the patient's field of view for much (or all) of the examination. One of the reasons that children often find it difficult to remain stationary while their eyes are being examined is that they find the examination process and instruments used intimidating. However, a decorative cover will help to give the instrument a non-threatening appearance, which is less likely to unnerve the child patient. The cover can be removed if the instrument is to be used to examine the eye of an adult, who may feel that the decorative appearance is inappropriate or even unsettling.

Thus, the invention is of particular advantage when the instrument is an ophthalmic instrument for use in examination of the eye. For example, the instrument may be a direct ophthalmoscope or a retinoscope.

However, the cover can be particularly advantageous if the instrument is an indirect ophthalmoscope as this is a relatively large instrument normally worn on the user's head as he or she looks into a patient's eye.

In this case, the cover preferably includes first aperture means for allowing light from the eye under examination to pass into viewing optics of the ophthalmoscope and to allow a beam of light generated by a light source in the ophthalmoscope housing to be directed into said eye.

Preferably, the cover includes further aperture means for allowing access to controls of the ophthalmoscope when the cover is in place. For example, the ophthalmoscope may have a forward projecting control lever for controlling, for example, the stereopsis of the ophthalmoscope, in which case the further aperture means may comprise an aperture from which the lever projects (when the cover is in place).

Preferably, the cover is resiliently retained on the housing by the engagement of latching elements on one of those two components with shoulders, preferably formed as part of recesses, on the other component.

The latching elements may conveniently be provided on (preferably formed integrally with) the cover, with the shoulders being provided in the housing.

Preferably, the cover is one of a number of such covers, of differing appearances, each of which is releasably attachable to the ophthalmic instrument, thereby to enable any selected one of the covers to be releasably retained on the instrument.

According to a second aspect of the invention, there is provided a decorative cover for an indirect ophthalmoscope as hereinabove described, the ophthalmoscope having a housing provided with one or more shoulders, wherein the cover is generally T-shaped to correspond to the shape of the housing, and includes at least one finger which is resiliently connected to the housing and which carries the latching formation for engaging said shoulder to enable the cover to be releasably retained on the housing.

The cover may be decorative in so far as it bears surface ornamentations, such as a printed pattern or design, or is decoratively shaped, or has a combination of both types of decorative feature.

### BRIEF DESCRIPTION OF THE DRAWINGS

The invention will now be described, by way of example only, with reference to the accompanying drawings in which:

FIG. 1 is a partially exploded isometric view of an indirect ophthalmoscope (including cover) in accordance with the invention;

FIGS. 2 and 3 are isometric views of part of the housing for the ophthalmoscope, and respectively show the exterior and interior of that housing part;

FIG. 4 is a further isometric view of the same part of the housing, with the cover attached thereto, and FIGS. 4A-4C are more detailed views of part of the cover and housing shown in FIG. 4; and

FIGS. 5 and 6 are respectively front and rear elevational views of the cover for the ophthalmoscope.

### DETAILED DESCRIPTION

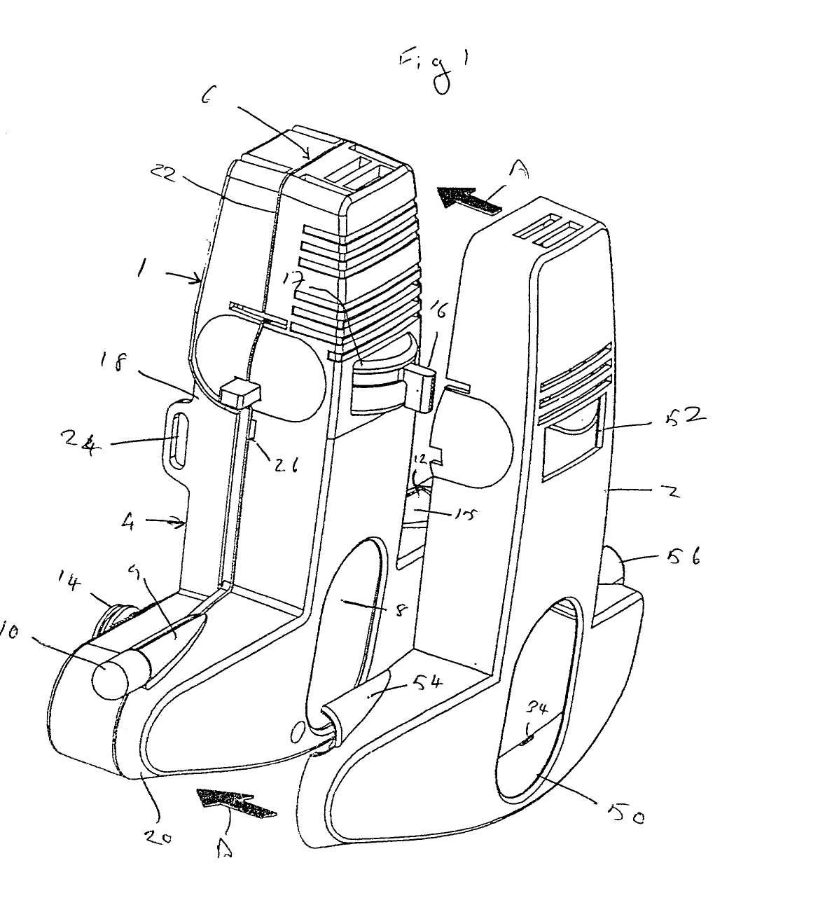

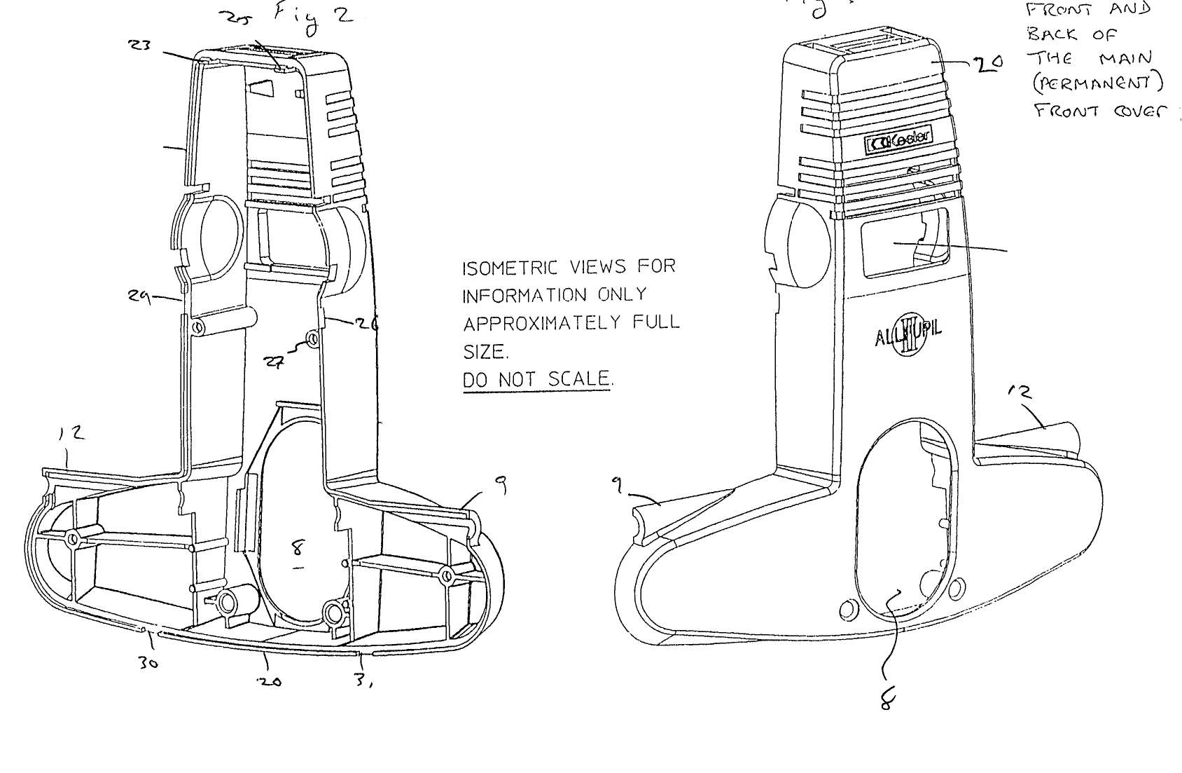

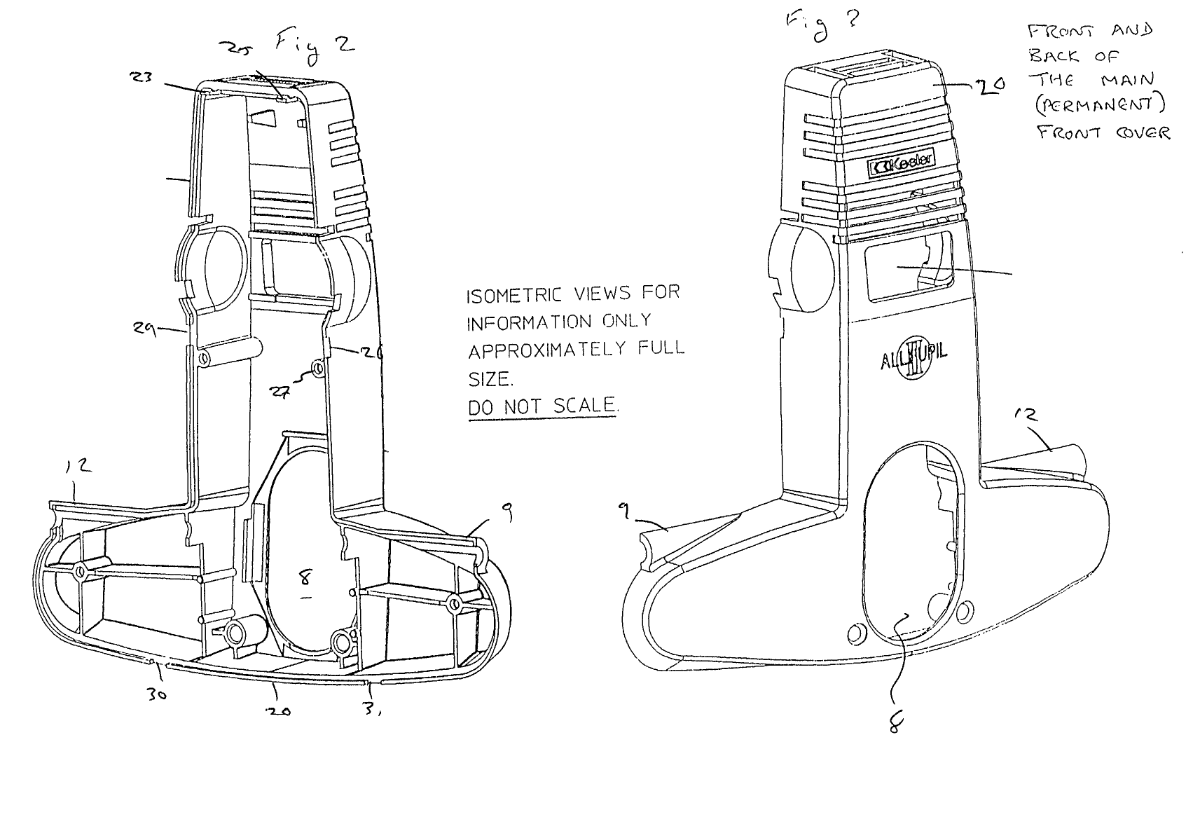

With reference to FIG. 1, an ophthalmoscope in accordance with the invention comprises a housing 1, which contains the components of the ophthalmoscope, and from which controls for operating the ophthalmoscope extend. The ophthalmoscope further comprises a front cover 2 which is releasably attachable to the housing 1.

The internal components and linking mechanisms of the ophthalmoscope are not shown or described in detail as they are substantially identical to the components of the present Applicants' known ophthalmoscopes.

The housing 1 is in substantially the shape of an inverted T having a stem 4, the upper portion 6 of which contains a light source positioned above an angled mirror further down the stem. The mirror is in registry with a front window 8 in the housing, and is arranged to reflect light from the source into an eye of a patient positioned in front of the ophthalmoscope. The mirror is mounted in the housing 1 via an adjustment mechanism which enables the angulation of the mirror to be adjusted by rotation of either of two control knobs 10 and 12 projecting horizontally from a region of the crosspiece of the T-shaped housing 1 adjacent the base of its stem.

The upper portion 6 of the housing also contains an adjustable aperture situated in the path of light from the source to the mirror. The user may adjust the size of the aperture using a slider control 16 to select a desired size of the patch of light to be projected into the patient's eye. The slider control 16 projects forwardly from a semi-circular guide 17 on the front of the top portion 6.

The mirror is positioned immediately above a set of viewing optics, which are situated within the crosspiece of the T-shaped housing 1, and which receive light from an eye under examination. Those optics include a pair of vertical mirrors situated immediately behind the window 8, and each of the mirrors is angled so as to reflect that light towards the respective adjacent end of the stem of the housing 1 to a further vertical mirror which reflects the light rearwardly and into a respective one of a left and right eyepiece through which the user views the image of the eye under examination. The right eyepiece is shown in FIG. 1 and is referenced 14.

As can be seen in FIG. 1, the housing 1 is of a two-part construction, being constituted by front and rear T-shaped portions 18 and 20 which join together so that the front of the portion 18 abuts the rear of the portion 20. The portions 18 and 20 are so sized and shaped that, where the two portions meet, there is defined a peripheral groove 22 extending around the sides, the top and the bottom of the housing 1.

The rear portion 18 also includes an integral elongated eyelet 24 situated on one side of the stem of the T. An identical eyelet (not shown) is situated on the opposite of the housing. The eyelets provide anchoring points for a headband assembly via which the ophthalmoscope can be attached to the user's head.

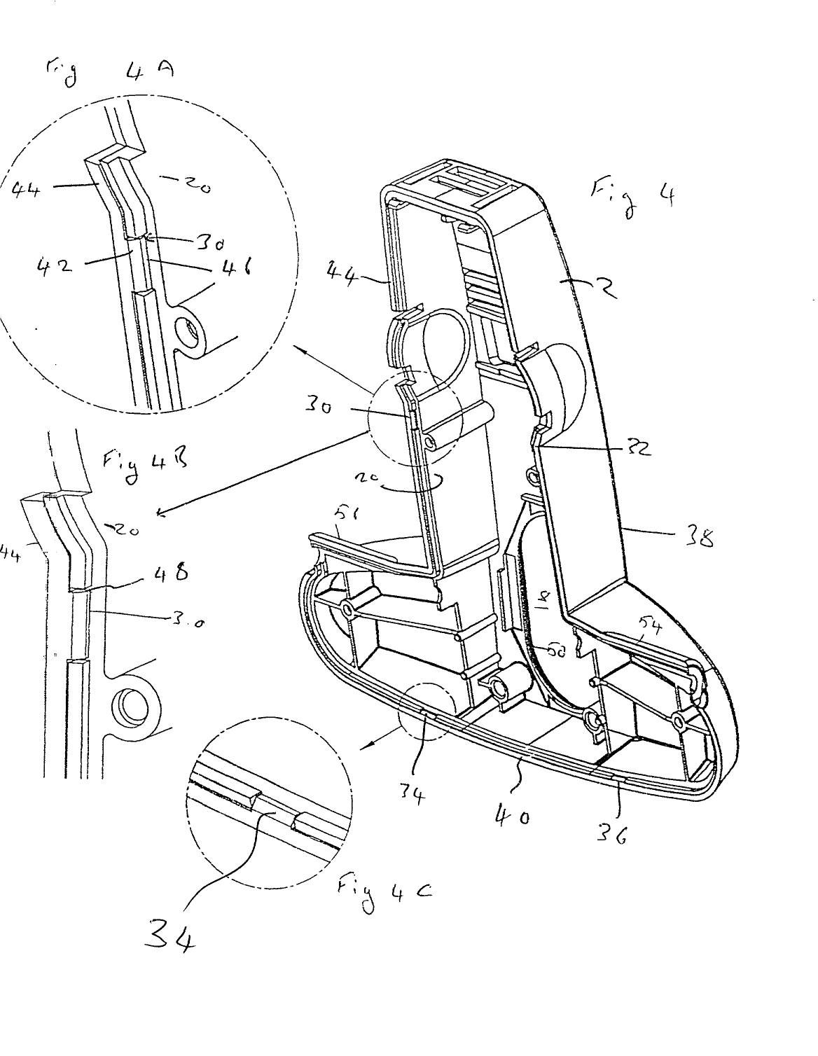

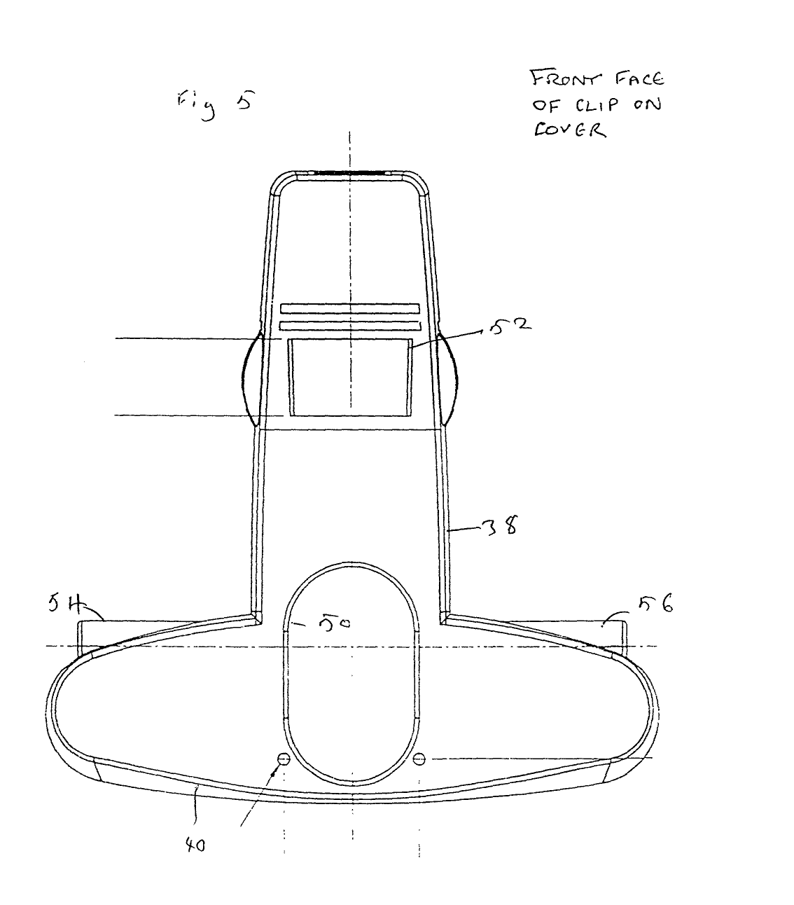

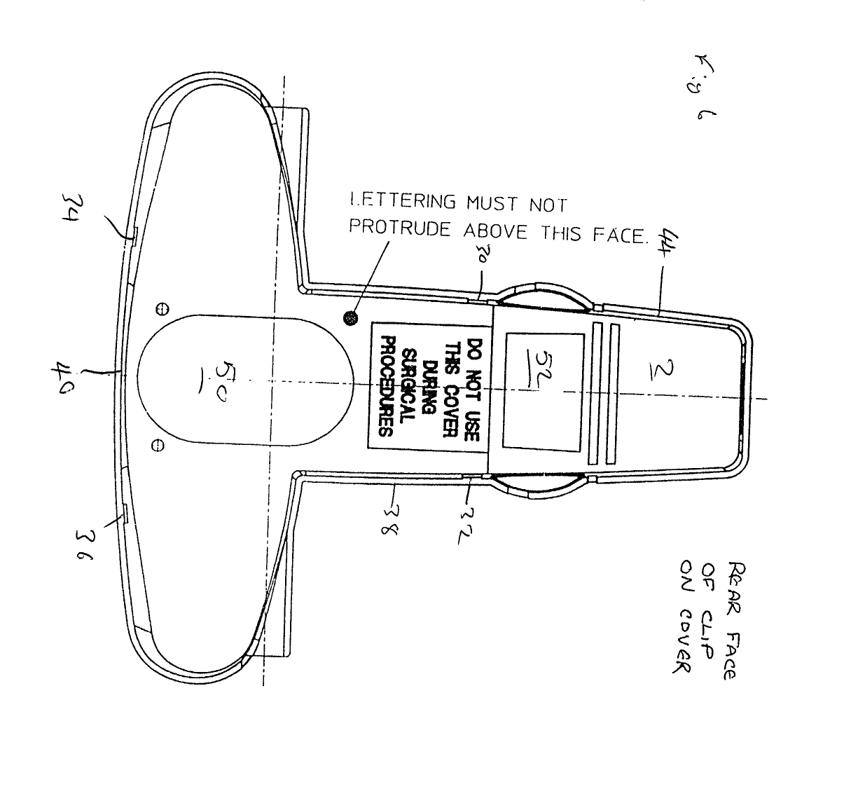

The front portion of the housing is formed with a number of part-rectangular rebates, 26, 29, 30, 31 about its edge. These rebates are situated adjacent the groove 22, and are in registry with corresponding latching hooks formed in the cover 2. The positions and details of the latching hooks are shown in FIGS. 4, 4A-4C and 6. The cover 2 has four CUSP latching hooks 30, 32, 34 and 36.

The portion 20 has upper latching fingers 23 and 25 and a series of screw holes (for example 27) which correspond to complementary formations on the portion 18, so that the portion 20 is attached to the portion 18 in a conventional manner.

The shape of the cover 2 is also substantially that of an inverted T with a stem 38 and a crosspiece 40. The latching hooks 30 and 32 are positioned at opposite sides of the stem portion 38, with the latching hook 32 being in registry with the rebate 26, and the latching hooks 34 and 36 are situated along the bottom of the crosspiece at positions equidistant from a vertical centre line of the T. Each hook is resiliently flexible, and is formed integrally with the cover 2, and has a forward cannning surface, for example surface 42, which extends inwardly from an exterior wall portion 44 of the cover 2. The camming surface 42 leads to a flat upper surface 46, and each hook has a rear surface 48 (FIG. 4B) that is substantially perpendicular to the plane of the wall 44, and hence the exterior of the cover 2.

Each of the latching hooks stands slightly proud of the adjacent portions of the cover wall, and the dimensions of the cover relative to the portion 20 of the housing are such that, as the cover is pushed onto the housing (in the direction indicated by the arrows A of FIG. 1), the camming surfaces 42 engage the portion 20, pushing the hooks 30 and 32 outwards and the hooks 34 and 36 downwards until each hook is in registry with its respective recess, whereupon the hooks connect into the recesses so that the rear surface of each hook engages the shoulder defined by the forward end of its respective recess. Thus, the latching hooks and recesses retain the cover 2 on the front portion 20 of the housing 1.

The cover 2 includes a first aperture means in the form of forward opening 50 which is in registry with the window 8 when the cover 2 is in place on the housing 1. When the cover 2 is in this position, a second aperture means, comprising opening 52 enables the lever 16, and the semi-circular guide 17 at the base of the lever 16, to extend forward of the cover 2 and to be accessible to the user.

The cover 2 also has a pair of opposed, part cylindrical portions 54 and 56 which fit over the front part of the sleeves 9 and 15 from which the knobs 10 and 12 protrude.

The groove 22 abuts the rear edge of the cover 2 so as to facilitate the location and retention of the cover 2 on the housing 1.

If the cover 2 is to be removed, this can be done simply by pulling the latching hooks outwards (using the fingernails or a small tool such as a screwdriver), so that they can be moved clear of the recesses.

The cover 2 may bear a decorative design (for example a picture of a cartoon character) and be interchangeable with other covers which have different designs. In addition, the ophthalmoscope can be used with or without the cover 2. Thus, the cover 2 could be used to make the ophthalmoscope look less threatening to a small child, but could be removed if the same ophthalmoscope is used to examine an adult's eyes.

1. An instrument for use in the direct examination of the human body or part thereof, the instrument having a cover so shaped as to allow the iustrument to be used with the cover in place, wherein the cover is detachable, so that the instrument can also be used without the cover.

2. An instrument according to claim 1, which the cover bears one or more distinctive identification indicia.

3. An instrument according to claim 1, in which the indicia comprises at least one of a printed pattern design or distinctively shaped portion.

4. An instrutment according to claim 1, in which the cover bears a decorative design.

5. An instrument according to claim 4, in which the instrument has a housing which contains one or more components thereof, wherein the cover extends over the front of the housing, when fitted thereon.

6. An instrument according to claim 5, in which the cover is co-extensive with the housing when fitted thereon.

7. An instrument according to claim 4, in which the instrument comprises a device for examining the human eye.

8. An instrument according to claim 7, in which the device comprises an indirect ophthalmoscope and a headset for mounting the ophthalmoscope on a user's head.

9. An instrument according to claim 8, in which the cover includes a first aperture for allowing light from the eye under examination to pass into viewing optics of the ophthalmoscope and to allow a beam of light generated by a light source in the ophthalmoscope housing to be directed into said eye.

10. An instrument according to claim 9, in which the cover includes a future aperture for allowing access to controls of the ophthalmoscope when the cover is in place.

11. An instrument according to claim 5, in which the cover is resiliently retainable on the housing by the engagement of latching elements on one of those two components with shoulders, preferably formed as part of recesses, on the other component.

12. An instrument according to claim 11, in which latching elements are provided on the cover, with the shoulders being provided in the housing.

13. An instrument according to claim 12, in which the latching elements are integrally formed with the cover.

14. An instrument according to claim 7, in which the cover is one of a number of such covers, of differing appearances, each of which is releasably attachable to the ophthalmic instrument, thereby to enable any selected one of the covers to be releasably retained on the instrument.

15. A decorative cover for an indirect ophthalmoscope as hereinabove described, the ophthalmoscope having a housing provided with one or more shoulders, wherein the cover. is generally T-shaped to correspond to the shape of the housing, and includes at least one finger which is resiliently connected to the housing and which carries the latching formation for engaging said shoulder to enable the cover to be releasably retained on the housing.

16. A cover according to claim 15, in which the cover is decorative in so far as it bears surface ornamentations, such as a printed pattern or design, or is decoratively shaped, or has a combination of both types of decorative feature.
